(19) United States
(12) Patent Application Publication
(10) Pub. No.: US 2005/0008687 A1
(43) Pub. Date: Jan. 13, 2005

(54) Metal-porphyrin-complex-embedded liposomes, production process thereof, and medicines making use of the same

(75) Inventors: Makoto Yuasa, Soka-shi (JP); Noriyoshi Matsukura, Tsukuba-shi (JP); Aritomo Yamaguchi, Yokohama-shi (JP); Hiroyoshi Kawakami, Hachioji-shi (JP); Shoji Nagaoka, Kamakura-shi (JP); Masahiko Abe, Noda-shi (JP); Hitoshi Takebayashi, Tsukuba-shi (JP); Aiko Horiuchi, Miura-gun (JP); Akihiko Ogata, Toda-gun (JP); Takeshi Sakaya, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Makoto Yuasa, Soka-shi (JP)

(21) Appl. No.: 10/788,263

(22) Filed: Mar. 1, 2004

(30) Foreign Application Priority Data
Jul. 7, 2003 (JP) 2003-193138
Jul. 7, 2003 (JP) 2003-193139

(51) Int. Cl. A61K 31/555; A61K 31/4439; A61K 31/409; A61K 9/127

(52) U.S. Cl. 424/450; 514/185; 514/338; 514/410

(57) ABSTRACT

An metalloporphyrin-complex-embedded liposome, comprising a cationic metalloporphyrin complex and a lipid having liposome forming ability is disclosed.

As metalloporphyrin-complex-embedded liposomes according to the present invention act on superoxide anion radicals (O₂⁻), and can surely lower their concentration, they can exhibit superb effects for the treatment of cancers and have excellent characteristics as antioxidants.

### TECHNICAL FIELD

This invention relates to metalloporphyrin-complex-embedded liposomes, and more specifically to metalloporphyrin-complex-embedded liposomes capable of acting as anticancer agents or antioxidants in the body, and also to their production process.

### BACKGROUND ART

Numerous reactive oxygen species formed in the body are generally considered to take part in the onset of many morbidities such as inflammatory diseases, neural diseases, arterial sclerosis, cancer and diabetes. In the body, however, there are radical scavenger enzymes such as superoxide dismutase (SOD), catalase and glutathione peroxidase against such reactive oxygen species to normally maintain a balance.

A great deal of superoxide anion radical (abbreviated as superoxide or O₂⁻.) is, however, known to exist in cancer cells in the body, so that reductions in the activities of these enzymes are suggested.

Concerning diseases such as inflammatory diseases, neural diseases, arterial sclerosis and diabetes, on the other hand, their causes are also considered to be attributable to disturbances in radical scavenger enzymes such as SOD, catalase and glutathione peroxidase and consequent increases in reactive species such as O₂⁻..

As a metalloporphyrin complex has been reported to exhibit high SOD activity, its administration into the body is expected to effectively scavenge reactive oxygen species led by O₂⁻. and hence, to protect the body from in vivo injury which would otherwise be caused by reactive oxygen.

However, administration of a metalloporphyrin complex by itself into the body involves potential problems from the standpoint of safety and effects. It is, therefore, the current circumstance that its use as a medicine has not been realized yet to date.

With the foregoing in view, the present invention has as an object thereof the provision of a means, which permits safe administration of a metalloporphyrin complex into the body and moreover, exhibition of the SOD activity possessed by the metalloporphyrin complex.

The present invention also has as other objects thereof the provision of an anticancer agent capable of selectively showing effects only against cancer cells as a substitute for anticancer agents side effects of which have become a serious problem, such as cisplatin (CDDP) and mitomycin C (MMC); and the provision of an antioxidant for treating non-cancer diseases onsets of which are considered to involve reactive oxygen species, such as inflammatory diseases, neural diseases, arterial sclerosis and diabetes.

### DISCLOSURE OF THE INVENTION

Taking as a target O₂⁻. existing in cancer cells, the present inventors have proceeded with various investigations to develop a means for lowering their concentration by making use of the SOD activity of a metalloporphyrin complex. As a result, it has been found that embedding of a metalloporphyrin complex in a liposome makes it possible to safely administer the metalloporphyrin complex into the body while possessing the excellent SOD activity and moreover, allows it to remain in blood, leading to the completion of the present invention.

Described specifically, the present invention provides a metalloporphyrin-complex-embedded liposome, comprising a cationic metalloporphyrin complex and a lipid having liposome forming ability. The metalloporphyrin-complex-embedded liposome may preferarbly be formed by using an ion complex comprising a cationic metalloporphyrin complex and an anionic surfactant.

The present invention also provides a process for producing a metalloporphyrin-complex-embedded liposome. The process comprises reacting a cationic metalloporphyrin complex and an anionic surfactant to form an ion complex, and then mixing and ultrasonicating the ion complex and a lipid having liposome forming ability.

The present invention further provides a medicine which comprises, as an active ingredient, a metalloporphyrin-complex-embedded liposome comprising an ion complex and a lipid having liposome forming ability. The ion complex is formed of a cationic metalloporphyrin complex and an anionic surfactant.

### BRIEF DESCRIPTION OF THE DRAWINGS

FIG. 1 is a diagrammatic illustration schematically showing the construction of a Pr-embedded liposome. FIG. 1 shows, from the left side, an ion complex formed of 1 molecule of MTnMPyP (n=2,4) and 4 molecules of a surfactant, an ion complex formed of 1 molecule of MTnMPyP (n=2, 4) and 1 molecule of the surfactant, and MPPIX-DMPyAm.

FIG. 2 is a graphic representation showing the results of an anticancer characteristic test of FeT2 MPyP, an ion complex system and a liposome system. In the graphic representation, square dots indicate the results on FeT2 MPyP, circular dots indicate the results on the ion complex system, and triangular dots indicate the results on the liposome system.

FIG. 3 is a graphic representation illustrating added concentration-versus-cell viability relationships in an anticancer characteristic test of FeT2 MPyP, FeT2 MPyP+1SAS and FeT2 MPyP+4SAS as ion complex systems, and CDDP and MMC as conventionally-known anticancer agents. In the graphic representation, triangles indicate the results on FeT2 MPyP, square dots indicate the results on the FeT2 MPyP+1SAS ion complex, rhombic dots indicate the results on the FeT2 MPyP+4SAS ion complex, squares indicate the results on CDDP, and rhombi indicate the results on MMC.

FIG. 4 is a graphic representation obtained by plotting added concentration-versus-cell viability relationships in an anticancer characteristic test of the ion complexes and conventionally-known anticancer agents in FIG. 3, FeT2 MPyP+1SAS-embedded DMPC liposome and FeT2 MPyP+4SAS-embedded DMPC liposome. In the graphic representation, circular dots indicate the results on FeT2 MPyP+1SAS-embedded DMPC liposome, triangular dots indicate the results on FeT2 MPyP+4SAS-embedded DMPC liposome, square dots indicate the results on FeT2 MPyP+1SAS ion complex, rhombic dots indicate the results on FeT2 MPyP+4SAS ion complex, squares indicate the results on CDDP, and rhombi indicate the results on MMC.

FIG. 5 is a graphic representation illustrating added concentration-versus-cell viability relationships in an anticancer characteristic test of FeT2 MPyP+40AS-embedded, mixed lipid D liposome and FeT2 MPyP+4SAS-embedded DMPC liposome. In the graphic representation, circles indicate the results on FeT2 MPyP+4OAS-embedded, mixed lipid D liposome, triangular dots indicate the results on the FeT2 MPyP+4SAS-embedded DMPC liposome, squares indicate the results on CDDP, and rhombi indicate the results on MMC.

### BEST MODES FOR CARRYING OUT THE INVENTION

The term “metalloporphyrin-embedded liposome” as used herein means that a metal porphyrin complex is integrated in a liposome-constituting lipid with the metalloporphyrin complex either extending at a part thereof out of the liposome membrane or enclosed in its entirety within the liposome membrane.

The metalloporphyrin-complex-embedded liposome according to the present invention comprises an ion complex, which is formed of a cationic metalloporphyrin complex and an anionic surfactant, and a lipid having liposome forming ability.

The ion complex, which is a constituent of the metalloporphyrin-complex-embedded liposome according to the present invention (hereinafter simply called “the Pr-embedded liposome”) and is formed of the cationic metalloporphyrin complex and the anionic surfactant, (hereinafter simply called “the ion complex”) is prepared by reacting the surfactant with the cationic metalloporphyrin complex.

The cationic metalloporphyrin, one of the constituents of the ion complex, contains as substituent groups thereof groups each of which contains a cationic nitrogen atom, and examples include those represented by the following formulas (I), (II) or (III): 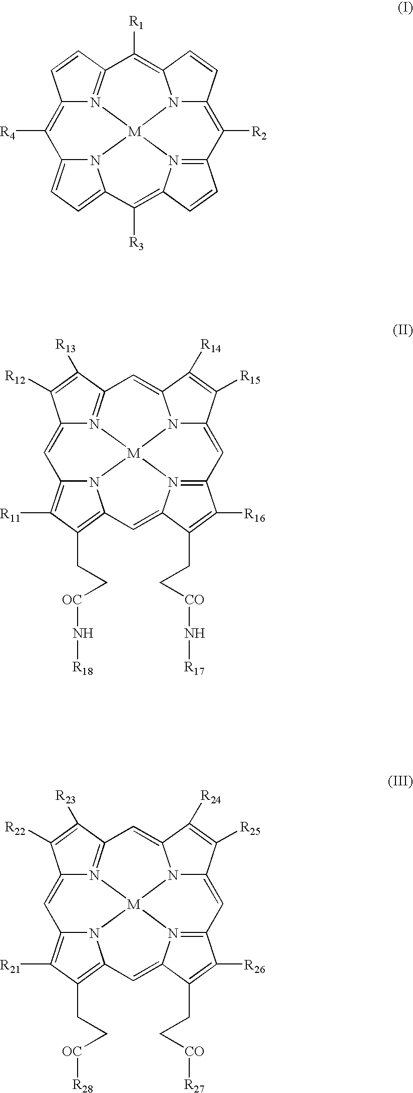
wherein R₁ to R₄ each independently represents a group selected from an N-(lower alkyl)pyridyl group, an N-(lower alkyl)-ammoniophenyl group and an N-(lower alkyl)imidazolyl group, R₁₁ to R₁₆ each independently represents a lower alkyl group or a lower alkoxy group, R₁₇ and R₁₈ each independently represents an N-(lower alkyl)pyridyl group, an N-(lower alkyl) ammoniophenyl group or an N-(lower alkyl) imidazolyl group, and R₂₁ to R₂₆ each independently represents a lower alkyl group or a lower alkoxy group, and R₂₇ and R₂₈ each independently represents an N-(lower alkyl)ammoniophenyl group.

Specific examples include those containing methylpyridyl groups as groups R₁ to R₄ in the formula (I), i.e., 5,10,15,20-tetrakis(2-methylpyridyl)porphyrin (T2 MPyP), 5,10,15,20-tetrakis(3-methylpyridyl)porphyrin, and 5,10,15,20-tetrakis(4-methylpyridyl)porphyrin (T4 MPyP); those containing ethylpyridyl groups as groups R₁ to R₄ in the formula (I), i.e., 5,10,15,20-tetrakis(2-ethylpyridyl)porphyrin, 5,10,15,20-tetrakis(3-ethylpyridyl)porphyrin, and 5,10,15,20-tetrakis(4-ethylpyridyl)porphyrin; those containing propylpyridyl groups as groups R₁ to R₄ in the formula (I), i.e., 5,10,15,20-tetrakis(2-propylpyridyl)porphyrin, 5,10,15,20-tetrakis(3-propylpyridyl)porphyrin, and 5,10,15,20-tetrakis(4-propylpyridyl)porphyrin; those containing butylpyridyl groups as groups R₁ to R₄ in the formula (I), i.e., 5,10,15,20-tetrakis(2-butylpyridyl)porphyrin, 5,10,15,20-tetrakis(3-butylpyridyl)porphyrin, and 5,10,15,20-tetrakis(4-butylpyridyl)porphyrin; those containing methylammoniophenyl groups as groups R₁ to R₄ in the formula (I), i.e., 5,10,15,20-tetrakis(2-methylammoniophenyl)porphyrin, 5,10,15,20-tetrakis(3-methylammoniophenyl)porphyrin, and 5,10,15,20-tetrakis(4-methylammoniophenyl)porphyrin; and those containing methylimidazolyl groups as groups R₁ to R₄ in the formula (I), i.e., 5,10,15,20-tetrakis(2-methylimidazolyl)porphyrin, 5,10,15,20-tetrakis(3-methylimidazolyl)porphyrin, and 5,10,15,20-tetrakis(4-methylimidazolyl)porphyrin.

Also included are one containing methyl groups as groups R₁₁, R₁₂, R₁₄ and R₁₆, vinyl groups as groups R₁₃ and R₁₅ and methylpyridyl groups as groups R₁₇ and R₁₈ in the formula (II), i.e., 1,3,5,8-tetramethyl-2,4-divinyl-6,7-di(methylpyridylamidoet hyl)porphyrin (PPIX-DMPyAm); one containing methyl groups as groups R₁₁, R₁₂, R₁₄ and R₁₆, vinyl groups as groups R₁₃ and R₁₅ and ammoniophenyl groups as groups R₁₇ and R₁₈ in the formula (II), i.e., 1,3,5,8-tetramethyl-2,4-divinyl-6,7-di(ammoniophenylamidoet hyl)porphyrin; one containing methyl groups as groups R₁₁, R₁₂, R₁₄ and R₁₆, vinyl groups as groups R₁₃ and R₁₅ and methylimidazolyl groups as groups R₁₇ and R₁₈ in the formula (II), i.e., 1,3,5,8-tetramethyl-2,4-divinyl-6,7-di(methylimidazolylamid oethyl)porphyrin; one containing methyl groups as groups R₁₁, R₁₂, R₁₄ and R₁₆, methoxy groups as groups R₁₃ and R₁₅ and methylpyridyl groups as groups R₁₇ and R₁₈ in the formula (II), i.e., 1,3,5,8-tetramethyl-2,4-dimethoxy-6,7-di(methylpyridylamido ethyl)porphyrin; one containing methyl groups as groups R₁, to R₁₆ and methylpyridyl groups R₁₇ and R₁₈ in the formula (II), i.e., 1,2,3,4,5,8-hexamethyl-6,7-di(methylpyridylamidoethyl)porph yrin; and one containing ethyl groups as groups R₁₁ to R₁₆ and methylpyridyl groups R₁₇ and R₁₈ in the formula (II), i.e., 1,2,3,4,5,8-hexaethyl-6,7-di(methylpyridylamidoethyl)porphyrin.

Further included are one containing methyl groups as groups R₂₁, R₂₂, R₂₄ and R₂₆, vinyl groups as groups R₂₃ and R₂₅, and methylammonio groups as groups R₂₇ and R₂₈ in the formula (III), i.e., [1,3,5,8-tetramethyl-2,4-divinyl-6,7-di(methylammoniocarbon ylethyl)porphyrin; one containing methyl groups as groups R₂₁, R₂₂, R₂₄ and R₂₆, methoxy groups as groups R₂₃ and R₂₅, and methylammonio groups as groups R₂₇ and R₂₈ in the formula (III), i.e., [1,3,5,8-tetramethyl-2,4-dimethoxy-6,7-di(methylammoniocarb onylethyl)porphyrin; one containing methyl groups as groups R₂₁-R₂₆ and methylammonio groups as groups R₂₇ and R₂₈ in the formula (III), i.e., [1,2,3,4,5,8-hexamethyl-6,7-di(methylammoniocarbonylethyl)-porphyrin; and one containing ethyl groups as groups R₂₁-R₂₆ and methylammonio groups as groups R₂₇ and R₂₈ in the formula (III), i.e., [1,2,3,4,5,8-hexaethyl-6,7-di(methylammoniocarbonyl-ethyl)porphyrin.

As metals (M) coordinated in these cationic porphyrin complexes, preferred are iron (Fe), manganese (Mn), cobalt (Co), copper (Cu), molybdenum (Mo), chromium (Cr) and iridium (Ir).

Syntheses of the metal-coordinated, cationic porphyrin complexes represented by the formula (I) out of the above-exemplified cationic porphyrin complexes can be conducted following the process disclosed inter alia in K. Kalyanasundaram, Inorg. Chem., 23,2453(1984), A. D. Adler et al., J. Inorg. Nucl. Chem., 32, 2443(1970), T. Yonetani et al., J. Biol. Chem., 245, 2988 (1970), or P. Hambright, Inorg. Chem., 15, 2314 (1976).

Further, syntheses of the metal-coordinated, cationic porphyrin complexes represented by the formula (II) or (III) out of the above-exemplified cationic porphyrin complexes can be conducted following the process disclosed inter alia in E. Tsuchida, H. Nishide, H. Yokoyama, R. Youngand C. K. Chang, Chem. Lett., 1984, 991.

Incidentally, the above-described
metal[5,10,15,20-tetrakis(2-methylpyridyl)porphyrins](MT2 MPyP) and metal[5,10,15,20-tetrakis(4-methylpyridyl)porphyrins](MT4 MPyP) have chemical structures as illustrated below.

Chemical structure of MT2 MPyP:

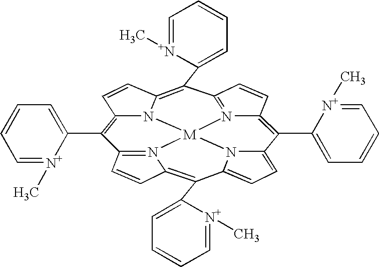

Chemical structure of MT4NPyP:

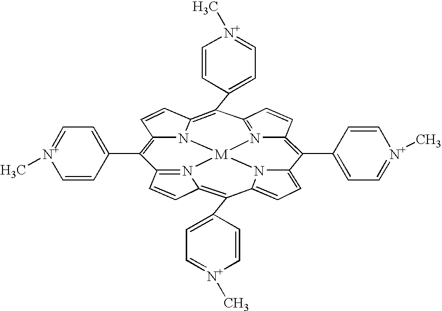

As the anionic surfactant as the other constituent forming each ion complex, on the other hand, an alkali metal salt of a fatty acid or an alkali metal salt of an alkylsulfuric acid is preferred. Illustrative are alkali metal salts of fatty acids such as lauric acid (LAS), myristic acid (MAS), palmitic acid (PAS), stearic acid (SAS) and oleic acid (OAS); and alkali metal salts of alkylsulfuric acids such as dodecylsulfuric acid (SDS), tetradecylsulfuric acid (STS), hexadecylsulfuric acid (SHS) and octadecylsulfuricacid (SOS). As the alkali metals in the alkali metal salts of fatty acids and alkylsulfuric acids, sodium, potassium and the like are preferred.

To form such an ion complex, it is only necessary to mix its corresponding cationic metalloporphyrin complex and an anionic surfactant in an appropriate solvent. The mixing ratio of the cationic metalloporphyrin complex to the anionic surfactant may be set at 1:1 to 1:20 or so in terms of molar ratio.

The ion complex formed as described above is then mixed with a lipid having liposome forming ability (hereinafter called “a lipid”), followed by the conversion into a Pr-embedded liposome by a method which is known per se in the art to form liposomes.

Examples of the lipid include phospholipids containing, as sole components, soybean lecithin (SBL), egg yolk lecithin (EYL), dilauroyl phosphatidylcholine (DLPC), dimyristoyl phosphatidylcholine (DMPC), dipalmitoyl phosphatidylcholine (DPPC), distearoyl phosphatidylcholine (DSPC), dioleoyl phosphatidylcholine (DOPC), monooleoyl-monoalkyl phosphatidylcholines (MOMAPC) and the like, respectively; and lipids containing these phospholipids as main components in combination with other components (which may hereinafter be called “mixed lipids”).

Examples of the components, which can be mixed with phospholipids upon preparation of such mixed phospholipids, include surfactants such as fatty acids, e.g., oleic acid (OAS) and surfactants, e.g., dimethylditetradecylammonium bromide (DTDAB), Tween-61 (TW61) and Tween-80 (TW80).

In particular, liposomes available from mixed lipid systems, which are composed of phospholipids such as DMPC and dipalmitoyl phosphatidylcholine (DPPC) and cationic surfactants such as dimethyldihexadecylammonium bromide (DHDAB), anionic surfactants such as OAS or SAS or nonionic surfactants such as TW61 and TW80, are pH-sensitive liposomes. As the pH is low, for example, in cancer cells, uptaking of such a liposome into the cancer cells results in deaggregation of the liposome so that a more effective sustained release of an anticancer agent is promoted. Systems with ion complexes embedded in such pH-sensitive liposomes (Pr-embedded pH-sensitive liposome) can be also synthesized.

Further, examples of the mixed lipids include those prepared by adding known cholesterols (Chol) to phospholipids and those prepared by adding polyethylene glycol or derivatives thereof to phospholipids.

To form the Pr-embedded liposome from the above-described ion complex and lipid, it is necessary as a first step to take these components in an appropriate solvent and then to mix them sufficiently.

Concerning the amounts of the ion complex and lipid to be used upon formation of the liposome, it is preferred to use the lipid in a proportion of from 10 to 500 moles, especially from 50 to 300 moles per mole of the ion complex.

The formation of the liposome can be conducted by a process already known in the art. For example, the above-described both components are dissolved and mixed in a volatile solvent, and thereafter, the volatile solvent alone is caused to evaporate off. A suitable aqueous solvent, for example, purified water, physiological saline or the like is then added to the residue, followed by vigorous stirring or ultrasonication into a Pr-embedded liposome.

Instead of such aqueous solvents, solutions with pharmaceutically-effective ingredients dissolved therein, certain culture media or the like can be used as needed. This makes it possible to obtain Pr-embedded liposomes with such solutions, media or the like enclosed therein.

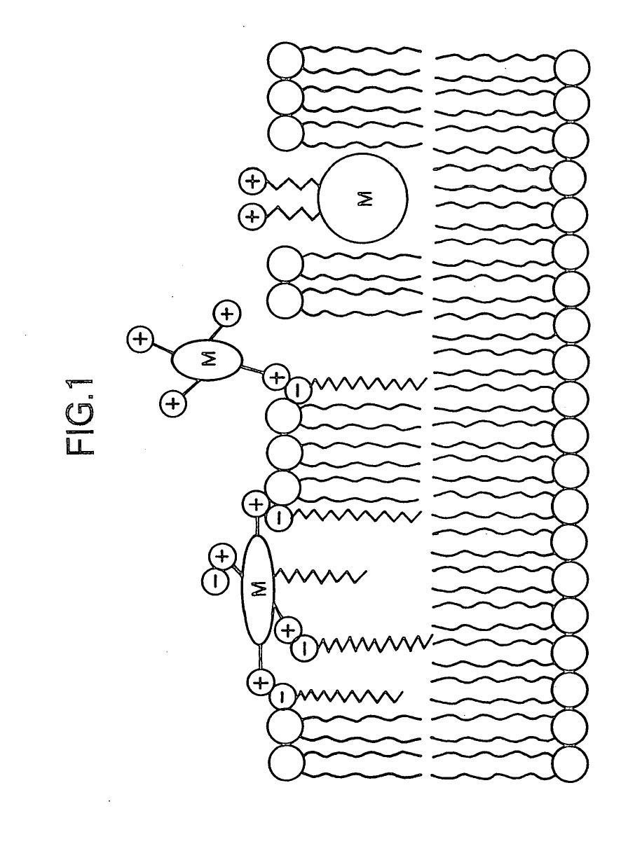

Structural analyses of Pr-embedded liposomes obtained as described above were performed by spectrofluorometry, dynamic light scattering analysis and the like as will be described subsequently herein. As a result, it has been found that as shown in FIG. 1, cationic metalloporphyrin complex parts exist on the surface of the liposome or in hydrophilic molecular groups such as the lipid while alkyl side chains of the surfactant are embedded in hydrophobic molecular areas of the lipid.

It has also found that the liposome has a vesicle size not greater than 100 nm and hence, that its size is small enough to reach cells when uptaken into the body.

Anticancer characteristic tests of Pr-embedded liposomes were also conducted as will be described subsequently herein. As a result, it has been demonstrated that the use of the Pr-embedded liposomes bring about better effects than the administration of simple cationic metalloporphyrin complexes which are raw materials for the liposomes, and also that their effects are far higher than those available from cisplatin or mitomycin C currently employed as an anticancer agent.

In addition, the Pr-embedded liposomes were also evaluated in SOD activity. It has been ascertained that they exhibit SOD activity as high as the simple cationic metalloporphyrin complexes as raw materials for the liposomes and accordingly, that they can be used as blood-residence-type, SOD mimics.

As described above, the Pr-embedded liposomes according to the present invention have excellent anticancer activities and are usable as anticancer agents in the field of clinical oncology.

It is, therefore, possible to treat the cancers of cancer patients by administering the Pr-embedded liposomes according to the present invention to the cancer patients by direct administration, intravenous administration, subcutaneous administration or the like.

The Pr-embedded liposomes according to the present invention are also equipped with superb antioxidation action, and can protect the body from in vivo injury which would otherwise be caused by reactive oxygen, such as inflammatory diseases, neural diseases, arterial sclerosis or diabetes.

By administering them to patients suffering from inflammatory diseases, neural diseases, arterial sclerosis or diabetes by direct administration, intravenous administration, subcutaneous administration or the like, these diseases of the patients can also be treated, accordingly.

### EXAMPLES

The present invention will hereinafter be described in further detail based on Examples and Tests, although the present invention shall by no means be limited by the following Examples.

### Example 1

Synthesis of iron[5,10,15,20-tetrakis(2-methylpyridyl)porphyrin](FeT2 MPyP)

(1) After heating propionic acid (500 mL) to 100° C. under stirring, 2-pyridylcarboxyaldehyde (15 mL, 0.158 mol) was added. Subsequently, pyrrole (12 mL, 0.173 mol) was added little by little dropwise by a syringe, and refluxing was conducted at 100° C. for 1 hour to effect cyclizing condensation. Subsequent to the reaction, the reaction mixture was allowed to cool down to room temperature, and the solvent was distilled off. Neutralization, washing and column chromatography (alumina basic type I, chloroform) were performed to afford 5,10,15,20-tetrakis(2-pyridyl)porphyrin as the target product [yield: 1.1 g, (4.4%)].

¹H-NMR δ_{H} (CDCl₃, ppm):
−2.82(2H, H in pyrrole NH), 7.72-9.14(16H, H in pyridine), 8.87(8H, H in pyrrole).

UV-vis λₘₐₓ (chloroform, m):
418, 513, 544, 586, 645.

FAB-Mass (m/z):
619, 620.
   (2) Under an argon (Ar) atmosphere, 5,10,15,20-tetrakis(2-pyridyl)porphyrin (0.2 g, 3.2×10⁻⁴ mol) obtained above in the procedure (1) was added to and dissolved in dimethylformamide (150 mL). Iron bromide (FeBr₂), which had been obtained from iron (0.2 g) and 48% hydrobromic acid (5 mL), was added further, followed by refluxing for 4 hours. Subsequent to the reaction, the reaction mixture was allowed to cool down to room temperature, and the solvent was distilled off.

Extraction and column chromatography (alumina basic type I, methanol) were conducted to afford iron[5,10, 15,20-tetrakis(2-pyridyl)porphyrin]as a precursor [yield: 0.21 g (94%)].

UV-vis λₘₐₓ (methanol, m):
408, 512, 566.

FAB-Mass (m/z):
672.
   (3) Into dimethylformamide (30 mL), iron[5,10,15,20-tetrakis(2-pyridyl)porphyrin](0.1 g) obtained above in the procedure (2) and methyl p-toluenesulfonate (6 mL) were added, and the resulting mixture was refluxed at 130° C. for 5 hours. Subsequent to the refluxing, the reaction mixture was allowed to cool down to room temperature, and the solvent was distilled off. Extraction and column chromatography (alumina basic type I, methanol) were conducted to afford FeT2 MPyP as the target substance (yield: 91%).

UV-vis λₘₐₓ (water, m):
408, 584.

Elemental analysis (%):

Found: C, 75.11; H, 3.97; N, 17.66, C/N 4.25. Calcd: C, 77.58; H, 4.24; N, 18.12, C/N 4.28.
(4) In a similar manner as in the above procedures (1) to (3) except that 2-pyridylcarboxyaldehyde was changed to 4-pyridylcarboxyaldehyde, iron[5,10,15,20-tetrakis(4-methylpyridyl)porphyrin](FeMT4 MPyP) was afforded. M[5,10,15-20-tetrakis(2-methylpyridyl)porphyrins](MT2 MPyP) and M[5,10,15,20-tetrakis(4-methylpyridyl)porphyrins](MT4 MPyP) (M: other metals) can also be synthesized following the above-described procedures.

### Example 2

Synthesis of iron[[1,3,5,8-tetramethyl-2,4-divinyl-6,7-di(4-methylpyridylamidoethyl)]porphyrin](FePPIX-DMPyAm)

A solution of iron[[1,3,5,8-tetramethyl-2,4-divinyl-6,7-di(carboxyethyl)]porphyrin] (500 mg, 8.1×10⁻⁴ mol) in a 10:1 mixed solvent of tetrahydrofuran and triethylamine (110 mL) was chilled, to which ethyl chloroformate (0.33 mL, 2.0×10⁻³ mol) was added, followed by a reaction for 90 minutes. 4-Aminopyridine (0.20 g, 2.0×10⁻³ mol) was then added, followed by a further reaction for 1 hour. Subsequently, the reaction mixture was allowed to stand overnight at room temperature.

After the solvent was distilled off, purification was conducted by column chromatography [silica gel, methanol/water (9/1)] and recrystallization to afford iron[[1,3,5,8-tetramethyl-2,4-divinyl-6,7-di(4-pyridylamidoethyl)]porphyrin] as a precursor (yield: 30 mg).

UV-vis λₘₐₓ (methanol, m):
398, 485, 596, 643.

FAB-Mass (m/z):
767.
   (2) The above-described precursor (30 mg, 3.9×10⁻⁵ mol) and methyl p-toluenesulfonate (0.75 mL) were dissolved in dimethylformamide (20 mL), followed by refluxing at 130° C. for 5 hours. The reaction mixture was allowed to cool down to room temperature, and the solvent was then distilled off. Purification was conducted by column chromatography (acidic alumina, methanol) to afford FePPIX-DMPyAm as the target substance (yield: 30 mg).

UV-vis λₘₐₓ (methanol, m):
398, 481, 579.

FAB-Mass (m/z):
797.
   (3) Using M[[1,3,5,8-tetramethyl-2,4-divinyl-6,7-di(carboxyethyl)]porphyrins](M: other metals) in place of iron[[1,3,5,8-tetramethyl-2,4-divinyl-6,7-di(carboxyethyl)]porphyrin] in the procedure (1), M[[1,3,5,8-tetramethyl-2,4-divinyl-6,7-di(4-methylpyridylamidoethyl)]porphyrins](MPPIX-DMPyAm)(M: other metals) can be synthesized likewise.

### Example 3

Synthesis of manganese[5,10,15,20-tetrakis(4-methylpyridyl)porphyrin](MnT4 MPyP)

(1) 4-Pyridylcarboxyaldehyde (15 mL) was added to propionic acid (500 mL), followed by heating. After the mixture had been heated to 100° C., pyrrole (12 mL) was added, and the thus-obtained mixture was refluxed for 1 hour. After the reaction, cooling, evaporation, neutralization and washing were conducted. Purification was then conducted by column chromatography (basic alumina, chloroform) to afford 5,10,15,20-tetrakis(4-pyridyl)porphyrin as purple crystals [yield: 1.68 g (7.08%)].

H-NMR δ_{H} (CDCl₃, ppm):
−2.9(2H, H in pyrrole NH), 8.2-9.1(16H, H in pyridine), 8.9(8H, H in pyrrole).

UV-vis λₘₐₓ (chloroform, m):
417, 513, 546, 589, 641.

FAB-Mass (m/z): 619, 620.

(2) After a solution of 5,10,15,20-tetrakis(4-pyridyl)porphyrin (100 mg) obtained above in the procedure (1) in dimethylformamide (100 mL) was next purged with argon (Ar), manganese acetate tetrahydrate (370 mg) was added, followed by refluxing for 3 hours under Ar. Subsequent to the reaction, cooling, evaporation, extraction, vacuum drying and the like were conducted to afford manganese[5,10,15,20-tetrakis(4-pyridyl)porphyrin] [yield: 81.8 mg (75.2%)].

UV-vis λₘₐₓ (chloroform, m):
477, 579, 611.

FAB-Mass (m/z):
672.
   (3) Thereafter, the manganese[5,10,15,20-tetrakis(4-pyridyl)porphyrin] (200 mg) and methyl p-toluenesulfonate (12 mL) were reacted at 120° C. for 5 hours. Subsequent to the reaction, cooling, extraction and the like were conducted, followed by the column chromatography [(1) acidic alumina and (2) basic alumina, methanol] to afford MnT4 MPyP as the target substance (yield: 153 mg).

UV-vis λₘₐₓ (water, m):
462, 559, 636.
   (4) In a similar manner as in the above procedures (1)-(3) except that 4-pyridylcarboxyaldehyde was changed to 2-pyridylcarboxyaldehyde in the procedure (1), manganese[5,10,15,20-tetrakis(2-methylpyridyl)porphyrin](MnMT2 MPyP) was afforded. M[5,10,15-20-tetrakis(4-methylpyridyl)porphyrins](MT4 MPyP) and M[5,10,15,20-tetrakis(2-methylpyridyl)porphyrins](MT2 MPyP) (M: other metals) can also be synthesized as in the above-described procedures.

### Example 4

Synthesis of manganese[[1,3,5,8-tetramethyl-2,4-divinyl-6,7-di(4-methylpyridylamidoethyl)]porphyrin](MnPPIX-DMPyAm)

(1) In accordance with the EC process(for example, E. Tsuchida, H. Nishide, H. Yokoyama, R. Young and C. K. Chang, Chem. Lett., 1984, 991, etc.), 1,3,5,8-tetramethyl-2,4-divinyl-6,7-di(carboxyethyl)porphyrin (protoporphyrin IX) (1 g) and ethyl chloroformate (2 mL) were reacted at 0° C. for 1 hour in tetrahydrofuran/triethylamine (250/3 mL) to yield an acid chloride.

The acid chloride and 4-aminopyridine (1.68 g) were reacted for 2 hours under the same conditions, and further, overnight at room temperature. After the reaction, evaporation and column chromatography [silica gel, methanol/chloroform (1/9)] were conducted to afford [1,3,5,8-tetramethyl-2,4-divinyl-6,7-di(4-pyridylamidoethyl)]porphyrin [yield: 0.469 g (68.4%)].

UV-vis λₘₐₓ (chloroform, m):
407, 506, 542, 575, 629.

FAB-Mass (m/z):
715.
   (2) After a solution of [1,3,5,8-tetramethyl-2,4-divinyl-6,7-di(4-pyridylamidoethyl)]porphyrin (200 mg) in dimethylformamide (200 mL) was next purged with Ar, manganese acetate tetrahydrate (686 mg) was added, followed by refluxing for 6 hours under Ar. Subsequent to the reaction, cooling, evaporation, washing, vacuum drying and the like were conducted to afford manganese[1,3,5,8-tetramethyl-2,4-divinyl-6,7-di(4-pyridylamidoethyl)]porphyrin [yield: 0.106 mg (45.7%)].

UV-vis λₘₐₓ (chloroform, m):
387, 465, 557, 621.
   (3) Further, the above-described manganese complex (200 mg) and methyl p-toluenesulfonate (9 mL) were reacted at 140° C. for 6 hours. Subsequent to the reaction, cooling, extraction and the like were conducted, followed by the column chromatography [acidic alumina, methanol] to afford MnPPIX-DMPyAm as the target substance [yield: 80.5 mg (37.5%)].

UV-vis λₘₐₓ (water, m):
389, 467, 556, 622.

### Example 5

Synthesis of Pr-Embedded Liposome (Part 1) FePPIX-DMPyAm (1 μmol) obtained in Example 2 and as a lipid, dimyristoyl phosphatidylcholine (DMPC) (200 μmol) were taken in a test tube, and then, a small amount of methanol was added, followed by mixing. After the solvent was distilled off to form a thin film, physiological saline (10 mL) was added to the test tube, and ultrasonication (under Ar, in an ice bath, 30 W, 30 min, probe ultrasonicator) was conducted. Subsequent to the ultrasonication, the resulting mixture was allowed to stand at room temperature for 1 hour and then sterilized by filtration (0.22 μm in diameter) to afford FePPIX-DMPyAm-embedded DMPC liposome (Invention Product 1).

Using MnPPIX-DMPyAm obtained in Example 4 and DMPC, MnPPIX-DMPyAm-embedded DMPC liposome (Invention Product 2) was afforded likewise.

### Example 6

Synthesis of Pr-Embedded Liposomes (Part 2)

(1) FeT2 MPyP (1.4 mg, 1 μmol), which is a cationic metalloporphyrin complex and was obtained in Example 1, and as a surfactant, SAS (0.3 mg, 1 μmol) were taken in a test tube, and then, methanol (5 mL) was added as a solvent. The resulting mixture was agitated to prepare an ion complex 1 (FeT2 MPyP+1SAS).

In addition, an ion complex 2 (FeT2 MPyP+4SAS) and an ion complex 3 (FeT2 MPyP+4OAS) were also obtained by using SAS (1.2 mg, 4 μmol) and OAS (1.2 mg, 4 μmol), respectively, in place of SAS (1 μmol).

Similarly, ion complexes 4-14 shown in Table 1 were each prepared by using FeT4 MPyP, MnT2 MPyP, MnT4 MPyP, FeT2 MPyP+MnT2 MPyP (molar ratio: 1:1) or FeT4 MPyP+MnT4 MPyP (molar ratio: 1:1) as a cationic metalloporphyrin complexe and SAS or OAS as a surfactant.

TABLE 1

| | | | Cationic |
|---|---|---|---|
| Ion | Cationic | | metalloporphyrin/ |
| complex | metalloporphyrin | Surfactant | surfactant |
| 1 | FeT2MPyP [1 μmol] | SAS [1 μmol] | 1/1 |
| 2 | FeT2MPyP [1 μmol] | SAS [4 μmol] | 1/4 |
| 3 | FeT2MPyP [1 μmol] | OAS [4 μmol] | 1/4 |
| 4 | FeT4MPyP [1 μmol] | SAS [1 μmol] | 1/1 |
| 5 | FeT4MPyP [1 μmol] | SAS [4 μmol] | 1/4 |
| 6 | FeT4MPyP [1 μmol] | OAS [4 μmol] | 1/4 |
| 7 | MnT2MPyP [1 μmol] | SAS [1 μmol] | 1/1 |
| 8 | MnT2MPyP [1 μmol] | SAS [4 μmol] | 1/4 |
| 9 | MnT4MPyP [1 μmol] | SAS [1 μmol] | 1/1 |
| 10 | MnT4MPyP [1 μmol] | SAS [4 μmol] | 1/4 |
| 11 | FeT2MPyP [0.5 μmol] + | SAS [1 μmol] | 1/1 |
| | MnT2MPyP [0.5 μmol] | | |
| 12 | FeT2MPyP [0.5 μmol] + | SAS [4 μmol] | 1/4 |
| | MnT2MPyP [0.5 μmol] | | |
| 13 | FeT4MPyP [0.5 μmol] + | SAS [1 μmol] | 1/1 |
| | MnT4MpyP [0.5 μmol] | | |
| 14 | FeT4MPyP [0.5 μmol] + | SAS [4 μmol] | 1/4 |
| | MnT4MPyP [0.5 μmol] | | |

(2) In a test tube, the above-described ion complex 1 (1 μmol) and as a lipid, DMPC (0.135 g, 200 82 mol) were taken, and a small amount of chloroform was added as a solvent, followed by mixing. After the solvent was distilled off to form a thin film, physiological saline (10 mL) was added to the test tube, and ultrasonication (under Ar, in an ice bath, 30 W, 30 min, probe ultrasonicator) was conducted. Subsequent to the ultrasonication, the resulting mixture was allowed to stand at room temperature for 1 hour and then sterilized by filtration (0.22 μm in diameter) to afford a Pr-embedded liposome (FeT2 MPyP+1SAS-embedded DMPC liposome; Invention Product 3).

Similarly, Pr-embedded liposomes [FeT2 MPyP+4SAS-embedded DMPC liposome (Invention Product 4); MnT4 MPyP+1SAS-embedded DMPC liposome (Invention Product 5); MnT4 MPyP+4SAS-embedded DMPC liposome (Invention Product 6)] were also afforded by using the ion complex 2 and DMPC, the ion complex 4 and DMPC, and the ion complex 5 and DMPC, respectively.

In addition, MnT4 MPyP+1SAS-embedded EYL liposome (Invention Product 7) was also obtained from the ion complex 4 and egg yolk lecithin (EYL).

### Example 7

Synthesis of Pr-Embedded pH-Sensitive Liposomes

(1) Using dimyristoyl phosphatidylcholine (DMPC), dimethylditetradecylammonium bromide (DTDAB), oleic acid (OAS), Tween-61 (TW61) and Tween-80 (TW80), mixed lipids A-D were prepared as shown in Table 2.

TABLE 2

| | Lipid composition (molar ratio) | | | | |
|---|---|---|---|---|---|
| Mixed lipid | DMPC | DTDAB | OAS | TW61 | TW80 |
| A | 75 | 75 | 50 | 2 | 0 |
| B | 75 | 75 | 50 | 0 | 2 |
| C | 160 | 20 | 20 | 0 | 2 |
| D | 180 | 10 | 10 | 0 | 2 |

(2) Using the mixed lipids A-D (202 μmol, each) shown in Table 2 and the ion complex 1 (1 μmol) obtained in Example 6, pH-sensitive, Pr-embedded liposomes (Invention Products 8-11) were prepared in a similar manner as in the procedure (2) of Example 6.

### Example 8

Synthesis of Pr-Embedded Liposomes (Part 4)

Using the ion complex 3 (1 μmol) shown in Table 1 and the lipids A, B and D (200 μmol, each) shown in Table 2 in the combinations as presented in Table 3, pH-sensitive, Pr-embedded liposomes (Invention Products 12-14) were prepared in a similar manner as in the procedure (2) of Example 6.

TABLE 3

| Metalloporphyrin- | | |
|---|---|---|
| complex-embedded | | |
| liposome solution | Ion complex | Lipid |
| Invention Product 12 | 3 | A |
| Invention Product 13 | 3 | B |
| Invention Product 14 | 3 | D |

### Example 9

Observation of Pr-Embedded Liposomes Under Transmission Electron Microscope (TEM)

To evaluate the shapes, vesicle size and the like of the Pr-embedded liposomes, their samples prepared by the freeze-fracture replica technique were observed under a transmission electron microscope (TEM) (“JEM-1200EX”, trade name; manufactured by JEOL, Ltd.). By the TEM observation of the FeT2 MPyP+4SAS-embedded DMPC liposome (Invention Product 4) obtained in Example 6, the formation of a liposome in the form of bilayer vesicles of not greater than 100 nm in vesicle size was confirmed. By a more detained examination, bilayer vesicles (liposome) having two vesicle size distributions, one having an average vesicle size of from about 20 to 30 nm and the other an average vesicle size of from about 50 to 60 nm, were observed.

### Example 10

Dynamic light Scattering Analysis of Pr-Embedded Liposomes (Part 1)

To determine the vesicle sizes and vesicle size distributions of the Pr-embedded liposomes, a dynamic light scattering analysis was conducted by a particle sizing system (“Nicomp 370”, trade name; manufactured by Pacific Scientific Corp.). For example, the dynamic light scattering analysis of FeT2 MPyP+4SAS-embedded DMPC liposome (Invention Product 4) synthesized in Example 6 confirmed the inclusion of two types of volume distributions consisting of 61.2% of vesicles having an average vesicle size of 24.6 nm and 38.8% of vesicles having an average vesicle size of 58.4 nm (in terms of number distributions, 94.5% of vesicles having an average vesicle size of 23.2 nm and 5.5% of vesicles having an average vesicle size of 52.5 nm). These results are consistent with the vesicle size distributions determined as a result of the TEM observation.

The results of the dynamic light scattering analysis of the Pr-embedded liposomes synthesized in Examples 5-6 are presented in Table 4.

TABLE 4

| | | 1^{st} Distribution | 2^{nd} Distribution |
|---|---|---|---|
| Porphyrin-complex-embedded liposome | | peak | peak |
| FeT2MPyP + 1SAS-embedded DMPC liposome | Volume distribution | 25.7 [67.7] | 90.3 [32.3] |
| (Invention Product 3) | Number distribution | 23.4 [98.7] | 79.2 [1.3] |
| FeT2MPyP + 4SAS-embedded DMPC liposome | Volume distribution | 24.6 [61.2] | 58.4 [38.8] |
| (Invention Product 4) | Number distribution | 23.2 [94.5] | 52.5 [5.5] |
| FeT2MPyP + 4OAS-embedded, mixed lipid A | Volume distribution | 27.3 [38.2] | 104.4 [61.8] |
| liposome (Invention Product 12) | Number distribution | 24.7 [96.6] | 89.0 [3.4] |
| FeT2MPyP + 4OAS-embedded, mixed lipid B | Volume distribution | 27.2 [63.1] | 94.5 [36.9] |
| liposome (Invention Product 13) | Number distribution | 25.6 [96.1] | 88.2 [3.9] |
| FeT2MPyP + 4OAS-embedded, mixed lipid D | Volume distribution | 35.4 [25.4] | 121.6 [74.6] |
| liposome (Invention Product 14) | Number distribution | 29.3 [95.8] | 101.9 [7.6] |

(Note)
The values outside the square brackets indicate vesicle sizes (nm), while the values inside the square brackets indicate distribution percentages.

From Table 4, it has become evident that each of the Pr-embedded liposomes has an average vesicle size smaller than 100 nm and, when administered into the body, can reach target cells beyond capillary endothelia.

### Example 11

Dynamic light Scattering Analysis of Pr-Embedded Liposomes (Part 2)

In a similar manner as in Example 10, a dynamic light scattering analysis was conducted on the MnT4 MPyP+1SAS-embedded DMPC liposome (Invention Product 5) and MnT4 MPyP+4SAS-embedded DMPC liposome (Invention Product 6) synthesized in Example 6.

As a result, it was found that the average vesicle size of Invention Product 5 was 29 nm (their distribution percentage was 99.8%, and as the remainder, vesicles having an average vesicle size of 173 nm amounted to approximately 0.2%) and also that the average vesicle size of Invention Product 6 was 29 nm (their distribution percentage was 99.7%, and as the remainder, vesicles having an average vesicle size of 171 nm amounted to approximately 0.3%). Therefore, each of them has been confirmed to have an average vesicle size smaller than 100 nm and is of a size small enough to cause no problem when administered into the body.

### Example 12

Spectroflurometry of Pr-Embedded Liposomes

(1) To ascertain at which positions of each Pr-embedded liposome the embedded molecules of the porphyrin complex existed in the liposome, spectrofluorometry of the Pr-embedded liposome was conducted by a spectrofluorometer (“RF-5300PC”, trade name; manufactured by Shimadzu Corporation).

As a metalloporphyrin complex generally causes fluorescence to extinct, cationic, metallofree porphyrin complexes into which the insertion of the metals had no been conducted (hereinafter called “metallofree complexes”) were synthesized in a similar manner as in Example 1, Example 2 or the like. In the analysis, those metallofree complexes were used as fluorescent probes instead of the metal porphyrin complexes. Synthesis of metallofree-complex-embedded liposomes, on the other hand, was conducted in a similar manner as in Examples 5-6 (As an abbreviation for a metallofree complex, the abbreviation for its corresponding cationic metalloporphyrin complex will hereinafter be used by replacing its “M” with “H₂”. For example, the metallofree complex corresponding to a metal[5,10,15,20-tetrakis(2-methylpyridyl)porphyrin](MT2 MPyP) will be referred to as “H₂T2 MPyP”, and the metallofree complex corresponding to a metal[5,10,15,20-tetrakis(4-methylpyridyl)porphyrin](MT4 MPyP) will be referred to as “H₂T4 MPyP”.

Spectrofluorometry (excitation wavelength: 456 nm, measurement wavelength range: 500 to 800 nm) of H₂T2 MPyP in various solutions containing a cationic metallofree complex-embedded liposome prepared as described above, for example, H₂T2 MPyP+4SAS-embedded DMPC liposome or H₂T2 MPyP+4SAS was performed. With an aqueous solution of H₂T2 MPyP+4SAS-embedded DMPC liposome, a fluorescence spectrum having a peak at 642 nm was obtained (relative fluorescence intensity at 642 nm: 43%). Fluorescence spectra of H₂T2 MPyP in various solvents such as methanol (47), ethanol (54), propanol (54), butanol (55) and ethyleneglycol (63) had similar spectrum profiles and intensities. In a fluorescence spectrum of H₂T2 MPyP in water, however, a peak around 642 nm was broadened and was significantly reduced in intensity (11).

As a consequence, H₂T2 MPyP embedded in a liposome is considered to exist in a polar environment similar to the above-described alcohols and hence, to exist around hydrophilic molecular groups of the bilayer membrane. Further, H₂T2 MPyP+1SAS-embedded DMPC liposome and H₂T2 MPyP+40AS-embedded mixed lipid D liposome gave similar results.

On the other hand, fluorescence spectra of H₂T4 MPyP in solutions of H₂T4 MPyP+4SAS-embedded DMPC liposome had peaks around 650 nm, and their peak intensities were between the fluorescence intensities of H₂T4 MPyP in water and methanol (fluorescence intensity: water<embedded liposome solution<methanol). As a consequence, H₂T4 MPyP in each solution of H₂T4 MPyP+4SAS-embedded DMPC liposome is determined to exist in an environment somewhat more nonpolar (somewhat more hydrophobic) than that existing in a water environment.

(2) Spectrofluorometry was then conducted by using 8-anilino-1-naphthalenesulfonic acid (ANS) as a fluorescent probe which exists around hydrophilic molecular groups of the bilayer membrane of each liposome and serves as an index for the polarity, fluidity and the like of the bilayer membrane. Spectrofluometry of ANS in methanol, methanol/chloroform and an ANS-embedded DMPC liposome (with no porphyrin complex embedded therein) solution gave fluorescence spectra, which were similar to one another and all presented a peak at 485 nm (excitation wavelength: 385 nm). A fluorescence spectrum of ANS in a solution of DMPC liposome with a porphyrin complex and ANS embedded together therein was next measured. Two peaks appeared at 450 and 500 nm, respectively, instead of 485 nm, and the fluorescence intensities of those two peaks were lower than that at 485 nm. Due to the existence of an absorption peak of the Soret band of the porphyrin complex around the two peaks, an interaction is considered to have taken place between ANS and the porphyrin complex. The porphyrin complex is hence considered to exist around ANS. With the foregoing in view, H₂T2 MPyP embedded in a liposome is considered to exist in a similar polar environment as in the above-described alcohols and to exist around hydrophilic molecular groups of the bilayer membrane.

### Example 13

Fluorescence Depolarization Measurement of Pr-Embedded Liposome (Part 1)

Using as a fluorescent probe 8-anilino-1-naphthalenesulfonic acid (ANS, 50 μM) existing around hydrophilic molecular groups of the bilayer membrane, a fluorescence depolarization measurement of a Pr-embedded liposome was conducted (polarimetry accessories for “RF-5300PC” and “RF-540/5000”, trade names, manufactured by Shimadzu Corporation; measurement temperature range: 5-45° C., excitation wavelength: 385 nm, fluorescence wavelength: 510 nm).

In a temperature-versus-polarization degree relationship ascertained by a florescence depolarization measurement of the ANS-containing DMPC liposome (blank), a decrease in the degree of fluorescence polarization was observed around a phase transition temperature (Tc=23° C.) of the bilayer membrane of the DMPC liposome. In a temperature-versus-degree relationship confirmed by a fluorescence depolarization measurement of ANS-containing Invention Product 3 (FeT2 MPyP+1SAS-embedded DMPC liposome), on the other hand, a decrease in the degree of fluorescence polarization was also observed around Tc as in the above-described case of the blank, but the degree of the decrease was smaller. This reduction in the degree of the decrease is based on an interaction between the FeT2 MPyP-SAS ion complex and DMPC, and supports that the ion complex exists in the bilayer membrane of the DMPC liposome. Further, pH-sensitive, FeT2 MPyP+40AS-embedded mixed lipid D liposome gave similar results.

### Example 14

Fluorescence Depolarization Measurement of Pr-Embedded Liposome (Part 2)

Using as a fluorescent probe ANS (50 μM) existing around hydrophilic molecular groups of the bilayer membrane, a fluorescence depolarization measurements of a Pr-embedded liposome was conducted (polarimetry accessories for “RF-5300PC” and “RF-540/5000¢, trade names, manufactured by Shimadzu Corporation; measurement temperature range: 5-45° C., excitation wavelength: 385 nm, fluorescence wavelength: 510 nm).

From a temperature-versus-polarization degree relationship (reverse sigmoidal curve) ascertained by a florescence depolarization measurement of the ANS-containing DMPC liposome (blank), the bilayer membrane of the DMPC liposome was found to have a gel-liquid crystal phase transition temperature (T_{c}) at about 23° C. A temperature-versus-polarization degree relationship confirmed by a fluorescence depolarization measurement of ANS-containing Invention Product 5 (MnT4 MPyP+1SAS-embedded DMPC liposome) shifted somewhat toward the side of lower temperatures, and the degrees of polarization plotted along the ordinates decreased in the gel range. These differences are based on an interaction between the MnT4 MPyP-SAS ion complex and DMPC, and support that the ion complex exists in the bilayer membrane of the DMPC liposome.

### Example 15

Anticancer Characteristic Test of Pr-Embedded Liposome (Part 1)

Anticancer characteristics of a Pr-embedded liposome according to the present invention were examined by a cytotoxicity test (apoptosis test) making use of the Alamar Blue technique.

Employed were the FeT2 MPyP+4SAS-embedded DMPC liposome system (Invention Product 4, FeT2 MPyP concentrations: 0, 12.5, 25, 50, 100 μM) as a test sample and its corresponding cationic metalloporphyrin complex (FeT2 MPyP) and ion complex system (FeT2 MPyP+4SAS) as reference samples. As cells, on the other hand, mouse lung cancer cells [Lewis Lung Carcinoma (LLC), Riken Gene Bank] were used.

In the test, the mouse lung cancer cells were cultured in DMEM medium with 10% FBS added therein. Subsequent to determination of the cell count and adjustment of the cell concentration, the resulting cell suspension was added to the individual wells of a 96-well plate (100 μL/well, cell count: 1×10⁴ cells/well), followed by incubation for 24 hours in a carbon dioxide incubator (CO₂:5%). After the medium was removed from the plate, sample solutions of the respective concentrations (100 μL/well, sample concentrations: 0 to 100 μM), said sample solutions having had been prepared in advance, were added, followed by further incubation for 24 hours in the CO₂ incubator.

An Alamar Blue solution, which had been sterilized by filtration, was added at 10 μL/well, followed by incubation for 5 hours. Subsequently, absorbance measurements (measurement wavelength: 570 nm, and reference wavelength: 600 nm) were conducted by using a microplate reader.

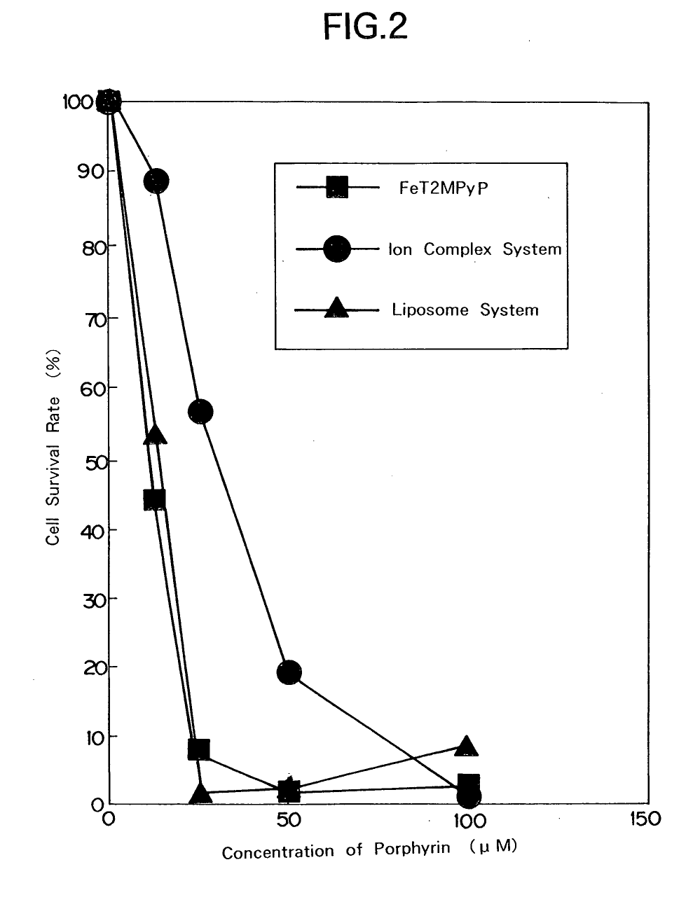

As a result, the Pr-embedded liposome (liposome system) according to the present invention, as illustrated in FIG. 2, exhibited better anticancer characteristics than the cationic metalloporphyrin complex (FeT2 MPyP) and the ion complex system.

### Example 16

Anticancer Characteristic Test of Pr-Embedded Liposomes (Part 2)

Anticancer characteristics of Pr-embedded liposomes according to the present invention were examined by a cytotoxicity test (apoptosis test) making use of the Alamar Blue technique as in Example 15.

As test samples, various Pr-embedded liposomes were used (metalloporphyrin complex concentrations: 0, 12.5, 25, 50, 100 μM). As reference samples, on the other hand, the components of the Pr-embedded liposomes, that is, the metalloporphyrin complexes (concentrations: 0, 12.5, 25, 50, 100 μM) and liposomes (concentrations: 2500, 5000, 10000, 20000 μM) were employed (the concentrations of both of the components were set corresponding to the concentrations of the Pr-embedded liposomes).

Provided as comparative samples were cisplatin (CDDP; concentrations: 0, 10, 20, 40, 80 μM) and mitomycin C (MMC; concentrations: 0, 7.5, 15, 30, 60 μM), which are anticancer agents employed at present. A test was conducted as in Example 15, and the following results were obtained.

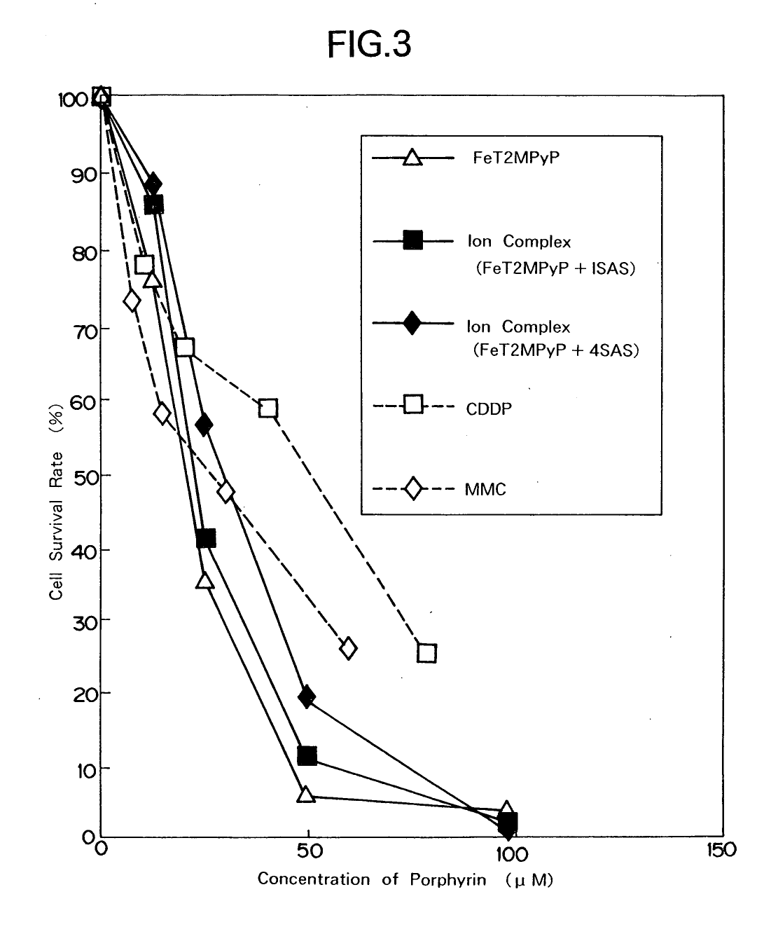

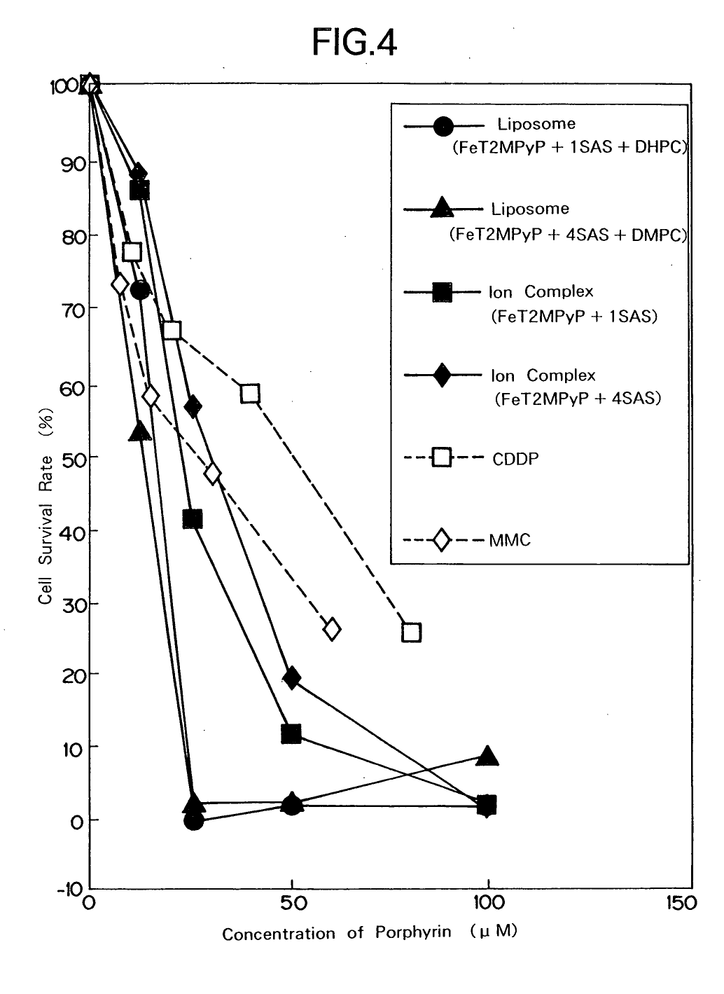

Firstly, the results on the systems making use of FeT2 MPyP as a metalloporphyrin complex are shown in FIG. 3 and FIG. 4. From FIG. 3, it is observed that in the case of each of FeT2 MPyP as a reference sample, FeT2 MPyP+1SAS and FeT2 MPyP+4SAS as ion complexes and CDDP and MMC as known anticancer agents, the viability of LLC dropped as the added concentration increased, and it is also shown that especially at FeT2 MPyP concentrations of 25 μM and higher, the viability of LLC in the case of each of FeT2 MPyP, FeT2 MPyP+1SAS and FeT2 MPyP+4SAS was lower than those in the cases of CDDP and MMC. From FIG. 4, on the other hand, it is appreciated that FeT2 MPyP+1SAS-embedded DMPC liposome (Invention Product 3) and FeT2 MPyP+4SAS-embedded DMPC liposome (Invention Product 4) as test samples had high cytotoxic activities and was superior to the FeT2 MPyP+1SAS ion complex and FeT2 MPyP+4SAS ion complex as reference samples and CDDP and MMC as known anticancer agents.

In the case of each of those FeT2 MPyP+1SAS-embedded DMPC liposome and FeT2 MPyP+4SAS-embedded DMPC liposome, the viability of LLC was observed to drop as the added concentration of the liposome increased, and the viability was 0% at the added concentrations of 25 μM and higher.

As is understood from the foregoing, the Pr-embedded liposomes, that is, FeT2 MPyP+1SAS-embedded DMPC liposome and FeT2 MPyP+4SAS-embedded DMPC liposome exhibit most effective anticancer characteristics compared with the ion complexes of the metalloporphyrin, the cationic metalloporphyrin complex and the currently-used anticancer agents (for example, the anticancer characteristics at 50 μM added concentration increased in the order of the currently-used anticancer agents<the ion complexes<the cationic metalloporphyrin complex<the Pr-embedded liposomes). As a consequence, the Pr-embedded liposomes are considered to be excellent anticancer agents.

### Example 17

Anticancer Characteristic Test of Pr-Embedded Liposomes (Part 3)

Anticancer characteristics of pH-sensitive, Pr-embedded liposomes were examined by a cytotoxicity test (apoptosis test) making use of the Alamar Blue technique as in Example 15.

As test samples, pH-sensitive, FeT2 MPyP+40AS-embedded mixed lipid D liposome (Invention Product 14) and FeT2 MPyP+4SAS-embedded DMPC liposome (Invention Product 4) (concentrations: 0, 12.5, 25, 50, 100 μM) were used. Employed as comparative samples, on the other hand, were cisplatin (CDDP; concentrations: 0, 10, 20, 40, 80 μM) and mitomycin C (MMC; concentrations: 0, 7.5, 15, 30, 60 μM), which are anticancer agents employed at present.

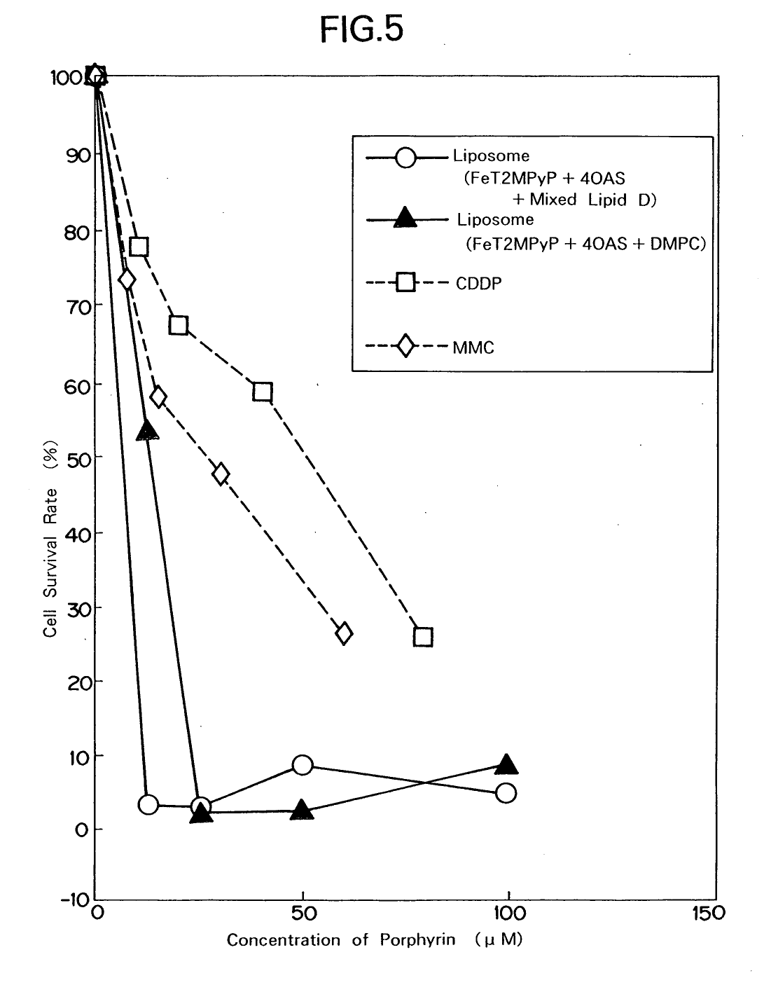

The results are shown in FIG. 5. FeT2 MPyP+40AS-embedded mixed lipid D liposome exhibited most effective anticancer characteristics, followed by FeT2 MPyP+4SAS-embedded DMPC liposome (for example, the anticancer characteristics at 12.5 μM added concentration increased in the order of CDDP and MMC<FeT2 MPyP+4SAS-embedded DMPC liposome<FeT2 MPyP+40AS-embedded mixed lipid D liposome).

Especially with FeT2 MPyP+40AS-embedded mixed lipid D liposome, the cell viability was substantially 0% even by its addition at a concentration as low as 12.5 μM. The cationic Pr-embedded liposomes according to the present invention have been found to be usable as excellent anticancer agents.

### Example 18

Interactions Between Metalloporphyrin Complexes and Hydrogen Peroxide (H₂O₂)

It has been reported that in the presence of large excess of hydrogen peroxide (H₂O₂), a low-molecular, metalloporphyrin complex is generally prone to decomposition because its porphyrin ring is exposed and undergoes interaction with H₂O₂ at high frequency [R. F. Pasternack and B. Halliwell, J. Am. Chem. Soc., 101, 1026 (1979)]. In this Example, interactions of MnT4 MPyP as a low-molecular metalloporphyrin complex, MnT4 MPyP+1SAS-embedded DMPC liposome (Invention Product 5) as a (high-molecular) liposome system and manganese[5,10,15,20-tetra(3-furyl)porphyrin][MnT3FuP]-embedded DMPC liposome* (comparative product) with H₂O₂ were investigated by UV-vis spectroscopy. Described specifically, the interactions were evaluated by measuring decay curves of the absorption peaks of Soret bands (463 nm) of the porphyrin complexes on the basis of their interactions with H₂O₂ and their decompositions and also by calculating their half-lives (t_{1/2}) from the decay curves. Incidentally, the results on copper/zinc superoxide dismutase (Cu/Zn-SOD) are also shown as a reference. The concentration of H₂O₂ was set at a large excess 1,000 times as much as the concentration of the corresponding metalloporphyrin complex. The results are shown in FIG. 5.

*A hydrophobic manganese-porphyrin complex embedded in hydrophobic molecular areas of the bilayer membrane (inside the bilayer membrane) of DMPC liposome.

TABLE 5

| Metalloporphyrin | |
|---|---|
| complex system | t_{1/2} (sec) |
| MnT4MPyP | 420 |
| MnT4MPyP + 1SAS-embedded DMPC | 570 |
| liposome (Invention Product 5) | |
| MnT3FuP-embedded DMPC liposome | 4100 |
| Cu/Zn-SOD | 10 |

As appreciated from Table 5, t_{1/2} increased in the order of Cu/Zn-SOD<MnT4 MPyP=MnT4 MPyP+1SAS-embedded DMPC liposome<MnT3FuP-embedded DMPC liposome. MnT4 MPyP is prone to decomposition as it is a low-molecular system and its porphyrin ring is exposed to undergo interaction with H₂O₂at high frequency, whereas MnT3FuP-embedded DMPC liposome is resistant to decomposition as it is a high-molecular system and its porphyrin ring is not exposed and does not undergo interaction with H₂O₂ at high frequency. However, the t_{1/2} of MnT4 MPyP+1SAS-embedded DMPC liposome is similar to that of MnT4 MPyP, and is 1/10 of the t_{1/2} of MnT3FuP-embedded DMPC liposome. This difference is considered to be attributable to a difference between the embedded position of MnT4 MPyP in the bilayer membrane of DMPC liposome and that of MnT3FuP in the bilayer membrane of DMPC liposome. Specifically, MnT4 MPyP is considered to exist in hydrophilic molecular groups of the bilayer membrane of the liposome (or in the vicinity of the surface layer) while MnT3FuP is considered to exist in the hydrophobic molecular areas. These results are consistent with those of Examples 12 and 14. Further, the t_{1/2} of MnT4 MPyP+1SAS-embedded DMPC liposome is greater than that of Cu/Zn-SOD, thereby also indicating the possession of higher H₂O₂ resistance than Cu/Zn-SOD.

### Example 19

Evaluation of SOD Activity of Pr-Embedded Liposomes (Part 1)

The SOD activity (in other words, O₂⁻. scavenging activity) of each Pr-embedded liposome was evaluated by the cytochrome c method proposed by Mccord and Fridovich or Butler et al. [(1) J. M. Mccord and I. Fridovich, J. Biol. Chem., 244, 6049 (1969) and (2) J. Butler, W. H. Kopenol, E. Margoliash, J. Biol. Chem., 257, 10747 (1982)]. Specifically, the evaluation was conducted as will be described hereinafter. Solutions (Solutions A) of each Pr-embedded liposome were prepared at five or more concentration levels of from 0 to 1,000 μM in terms of the concentration of the metalloporphyrin. Next, a 0.3 mM aqueous solution of xanthine, a 60 μM aqueous solution of cytochrome c and an aqueous, 30 mM phosphated buffer solution of pH 7.8 were each taken in an amount of 20 mL, followed by the addition of purified water (24 mL) to obtain a mixed solution (Solution B). To Solution B (20.1 mL), one of Solution A (0.3mL) and purified water (0.2 mL) were added, and the resulting mixture was allowed to stand at 25° C. for 10 min. With the resultant mixture, a 7 μg/mL aqueous catalase solution (0.1 mL) and a 25 U/mL aqueous xanthine oxidase (XOD) solution (0.3 mL) were promptly mixed, and UV-vis was measured with time at 550 nm (absorption peak based on the formation of ferrocytochome c)(the final concentrations of the respective components in the test solution were as follows: the metal porphyrin complex, 0 to 100 SM; xanthine, 0.05 mM; XOD, 2.5U/mL; cytochrome c, 10 μM; catalase, 0.23 μg/mL). In addition, a similar measurement was also conducted on a system not added with any Pr-embedded liposome (blank). From “time-versus-absorbance at 550 nm” relationships as determined by the UV-vis spectroscopy with time, the formation rates (vᵢ and vₒ) of ferrocytochome c in the system not added with any Pr-embedded liposome and in the systems added with the Pr-embedded liposomes were determined, and further, inhibition coefficiencies (IC) were calculated in accordance with the below-described formula. Finally, the concentration (IC₅₀) of each metalloporphyrin complex at IC=50% was determined from the concentration of metalloporphyrin-versus-IC” relationship, and the IC₅₀ was used as an index of the SOSD-activating effect of the metalloporphyrin complex (smaller IC₅₀ indicates higher SOD activity). Incidentally, SOD activity was also evaluated with respect to each of the corresponding ion complexes (MnT4 MPyP+1SAS and MnT4 MPyP+4SAS) as a reference.
Inhibition coefficiency (IC)=1−(vᵢ/vₒ)
v₀: Formation rate of ferrocytochrome c in the system not added with any Pr-embedded liposome, and
vᵢ: Formation rate of ferrocytochrome c in a system added with a Pr-embedded liposome.

The IC₅₀ values of various metalloporphyrin complex systems are shown in Table 6. As the IC₅₀ of MnT4 MPyP, the literature value reported by Fridovich et al. is reproduced [I. Batinic-Haberle, L. Benov, and I. Fridovich, J. Biol. Chem., 273, 24251 (1998)].

TABLE 6

| Metalloporphyrin complex system | IC₅₀ (μM) |
|---|---|
| MnT4MPyP + 1SAS-embedded DMPC | 1.12 |
| liposome (Invention Product 5) | |
| MnT4MPyP + 4SAS-embedded DMPC | 1.14 |
| liposome (Invention Product 6) | |
| MnT4MPyP + 1SAS | 0.97 |
| MnT4MPyP | 0.7 |

It is understood from the above results that MnT4 MPyP+1SAS-embedded DMPC liposome and MnT4 MPyP+4SAS-embedded DMPC liposome had IC₅₀ values similar to those of the low-molecular systems [MnT4 MPyP (literature value) and MnT4 MPyP+1SAS] and exhibited high SOD activity.

### Example 20

Evaluation of SOD Activity of Pr-Embedded Liposomes (Part 2)

The SOD activity (in other words, O₂⁻. scavenging activity) of each Pr-embedded liposome was evaluated by the stopped-flow method proposed by Riley et al. [D. P. Riley, W. L. Rivers, and R. H. Weiss, Anal. Biochem., 196, 344 (1991)]. Specifically, the evaluation was conducted as will be described hereinafter. At 36° C., a solution of potassium superoxide as an O₂⁻. production source in dimethylsulfoxide and one of 60 mM HEPES/HEPESNa buffered solutions (pH 8.1), which contained one of the Pr-embedded liposomes at various concentrations, were promptly mixed, and the decay in absorbance at 245 nm due to O₂⁻. (the decay curve of O₂⁻. scavenging reaction) was measured with time. From the decay curve, a “ln (absorbance)-versus-time” relationship was determined, and further, an apparent rate constant was calculated from the slope of the relationship. From the slope of the “concentration of metalloporphyrin complex-versus-apparent rate constant” relationship, the rate constant (K_{cat}) of the O₂⁻. scavenging reaction was finally determined. As a reference, ion complexes (MnT4 MPyP+1SAS and MnT4 MPyP+4SAS) were also evaluated likewise in SOD activity.

The K_{cat} values of various metalloporphyrin complex systems are shown in Table 7. As the K_{cat} of MnT4 MPyP, the literature value reported by Ohse, Kawakami et al. is reproduced [T. Ohse, S, Nagaoka, Y. Arakawa, H. Kawakami, and K. Nakamura, J. Inorg. Biochem., 85, 201 (2001)].

TABLE 7

| Metalloporphyrin complex system | K_{cat} (M⁻¹s⁻¹) |
|---|---|
| MnT4MPyP + 1SAS-embedded DMPC | 2.0 × 10⁷ |
| liposome (Invention Product 5) | |
| MnT4MPyP + 4SAS-embedded DMPC | 2.0 × 10⁷ |
| liposome (Invention Product 6) | |
| MnT4MPyP + 1SAS-embedded EYL | 1.5 × 10⁷ |
| liposome (Invention Product 7) | |
| MnT4MPyP + 1SAS | 1.9 × 10⁷ |
| MnT4MPyP + 4SAS | 1.9 × 10⁷ |
| MnT4MPyP | 2.2 × 10⁷ |

It is understood from the above results that MnT4 MPyP+1SAS-embedded DMPC liposome, MnT4 MPyP+4SAS-embedded DMPC liposome and MnT4 MPyP+1SAS-embedded EYL liposome had K_{cat} values close to those of the low-molecular systems [MnT4 MPyP (literature value), MnT4 MPyP+1SAS and MnT4 MPyP+4SAS] and exhibited high SOD activity.

### Example 21

Evaluation of SOD Activity of Pr-Embedded Liposomes (Part 3)

Various metalloporphyrin complexes, which represent Pr-embedded liposomes, were compared in SOD activity. MnT4 MPyP+1SAS-embedded DMPC liposome (MnT4 MPyP/liposome system), MnT3FuP-embedded DMPC liposome (MnT3FuP/liposome system) and MnT4 MPyP were used as samples, and were evaluated by the cytochrome c method and the stopped-flow method. Incidentally, the samples were prepared and measured as in Examples 19 and 20.

The K_{cat} and IC₅₀ values as indexes of the SOD activity of the various metalloporphyrin complexes are shown in Table 8.

TABLE 8

| | K_{cat} (M⁻¹s⁻¹) | IC₅₀ |
|---|---|---|
| MnT4MPyP/liposome system | 2.0 × 10⁷ | 1.12 μm |
| MnT4MPyP | 2.2 × 10⁷ | 0.74 μm |
| MnT3FuP/liposome system | 1.5 × 10⁶ | 12.0 μm |

The K_{cat} and IC₅₀ values of MnT4 MPyP are substantially consistent with its literature values shown in Table 7 and Table 6, and substantiate the validity of these evaluations. Further, K_{cat} increases in the order of the MnT3FuP/liposome system (MnT3FuP-embedded DMPC liposome)<the MnT4 MPyP/liposome system (MnT4 MPyP+1SAS-embedded DMPC liposome)=MnT4 MPyP, and IC₅₀ decreases in the order of the MnT3FuP/liposome system (MnT3FuP-embedded DMPC liposome)>the MnT4 MPyP/liposome system (MnT4 MPyP+1SAS-embedded DMPC liposome)=MnT4 MPyP. Accordingly, these two evaluation methods conform with each other. The K_{cat} and IC₅₀ of the MnT4 MPyP/liposome system (MnT4 MPyP+1SAS-embedded DMPC liposome) are similar to those of MnT4 MPyP, but are dissimilar to those of the MnT3FuP/liposome system (MnT3FuP-embedded DMPC liposome). These results are consistent with the results of Examples 16, 17 and 18. Anyhow, these results indicate that the MnT4 MPyP/liposome systems (MnT4 MPyP+1SAS-embedded DMPC liposome, MnT4 MPyP+4SAS-embedded DMPC liposome and MnT4 MPyP+1SAS-embedded EYL liposome) exhibit high SOD activity and are usable as effective antioxidants.

### INDUSTRIAL APPLICABILITY

The Pr-embedded liposomes according to the present invention act on superoxide anion radicals (O₂⁻.), and can surely lower their concentration.

Therefore, they can exhibit superb effects for the treatment of cancers and moreover, their effects are selective, so that they are usable as new anticancer agents free of side effect.

Further, the Pr-embedded liposomes according to the present invention are equipped with excellent characteristics as antioxidants such that they have SOD activity and they can remain in blood. They can, hence, protect the body from in vivo damage which would otherwise be caused by reactive oxygen.

1. A metalloporphyrin-complex-embedded liposome, comprising a cationic metalloporphyrin complex and a lipid having liposome forming ability.

2. A metalloporphyrin-complex-embedded liposome according to claim 1, wherein said cationic metalloporphyrin complex exists in a state forming an ion complex with an anionic surfactant.

3. A metalloporphyrin-complex-embedded liposome according to claim 1, wherein said cationic metalloporphyrin complex is represented by the following formula (I), (II) or (III): 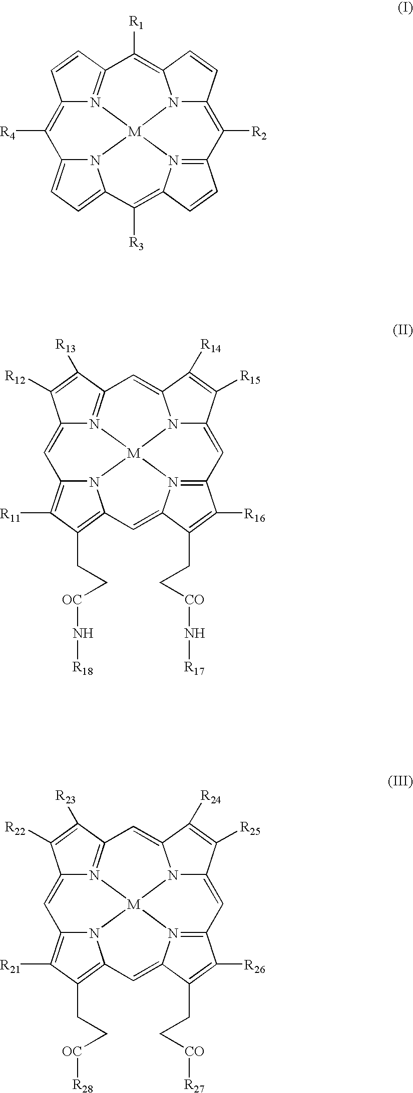
wherein R₁ to R₄ each independently represents a group selected from N-(lower alkyl)pyridyl groups, N-(lower alkyl)ammoniophenyl groups and N-(lower alkyl)imidazolyl groups, R₁, to R₁₆ each independently represents a lower alkyl group or a lower alkoxy group, R₁₇ and R₁₈ each independently represents an N-(lower alkyl)pyridyl group, an N-(lower alkyl) ammoniophenyl group or an N-(lower alkyl) imidazolyl group, and R₂₁ to R₂₆ each independently represents a lower alkyl group or a lower alkoxy group, and R₂₇ and R₂₈ each independently represents an N-(lower alkyl)ammoniophenyl group.

4. A metalloporphyrin-complex-embedded liposome according to claim 1, wherein said cationic metalloporphyrin complex comprises at least one of metal[5,10,15,20-tetrakis(2-methylpyridyl)porphyrins](MT2 MPYP), metal[5,10,15,20-tetrakis(4-methylpyridyl)porphyrins](MT4 MPyP) and metal[[1,3,5,8-tetramethyl-2,4-divinyl-6,7-di(4-methylpyridylamidoethyl)]porphyrins](MPPIX-DMPyAm), and the metal elements in said complexes are each independently selected from the group consisting of iron (Fe), manganese (Mn), cobalt (Co), copper (Cu), molybdenum (Mo), chromium (Cr) and iridium (Ir).

5. A metalloporphyrin-complex-embedded liposome according to claim 2, wherein said anionic surfactant is selected from the group consisting of alkali metal salts of lauric acid, myristic acid, palmitic acid, stearic acid, oleic acid, dodecylsulfuric acid, tetradecylsulfuric acid, hexadecylsulfuric acid and octadecylsulfuric acid.

6. A metalloporphyrin-complex-embedded liposome according to claim 1, wherein said lipid having liposome forming ability is a phospholipid.

7. A metalloporphyrin-complex-embedded liposome according to claim 1, wherein said lipid having liposome forming ability comprises at least one phospholipid selected from the group consisting of soybean lecithin (SBL), egg yolk lecithin (EYL), dilauroyl phosphatidylcholine (DLPC), dimyristoyl phosphatidylcholine (DMPC), dipalmitoyl phosphatidylcholine (DPPC), distearoyl phosphatidylcholine (DSPC), dioleoyl phosphatidylcholine (DOPC) and monooleoyl-monoalkyl phosphatidylcholines (MOMAPC).

8. A metalloporphyrin-complex-embedded liposome according to claim 1, wherein said lipid having liposome forming ability is a mixture of a phospholipid and a cholesterol.

9. A metalloporphyrin-complex-embedded liposome according to claim 1, wherein said lipid having liposome forming ability is a mixture of a phospholipid and polyethylene glycol or a derivative thereof.

10. A metalloporphyrin-complex-embedded liposome according to claim 1, wherein said lipid having liposome forming ability is a mixture of a phospholipid and a surfactant selected from the group consisting of OAS, dimethylditetradecylammonium bromide (DTDAB), Tween-61 (TW61) and Tween-80 (TW80).

11. A metalloporphyrin-complex-embedded liposome according to claim 1, which has a vesicle size not greater than 100 nm.

12. A process for producing a metalloporphyrin-complex-embedded liposome, which comprises reacting a cationic metalloporphyrin complex and an anionic surfactant to form an ion complex, and then mixing and ultrasonicating said ion complex and a lipid having liposome forming ability.

13. A medicine comprising as an active ingredient a metalloporphyrin-complex-embedded liposome comprising an ion complex and a lipid having liposome forming ability, said ion complex being formed of a cationic metalloporphyrin complex and an anionic surfactant.

14. A medicine according to claim 13, which is an anticancer agent.

15. A medicine according to claim 13, which is an antioxidant.

16. A medicine according to claim 13, which is a therapeutic drug for inflammatory diseases, neural diseases, arterial sclerosis or diabetes.

17. A treatment method of a cancer, which comprises administering to a cancer patient a metalloporphyrin-complex-embedded liposome comprising an ion complex and a lipid having liposome forming ability, said ion complex being formed of a cationic metalloporphyrin complex and an anionic surfactant.

18. A treatment method according to claim 17, wherein said administration is effected by direct administration, intravenous administration or subcutaneous administration.

19. A treatment method of an inflammatory disease, a neural disease, arterial sclerosis or diabetes, which comprises administering to its patient a metalloporphyrin-complex-embedded liposome comprising an ion complex and a lipid having liposome forming ability, said ion complex being formed of a cationic metalloporphyrin complex and an anionic surfactant.
